(19) United States
(12) Patent Application Publication
(10) Pub. No.: US 2024/0325385 A1
(43) Pub. Date: Oct. 3, 2024

(54) ANTI-TUMOR PHARMACEUTICAL COMPOSITION COMPRISING EZH2 INHIBITOR AND SCD1 INHIBITOR AND USE THEREOF

(71) Applicant: PEKING UNIVERSITY THIRD HOSPITAL, BEIJING (CN)

(72) Inventors: Lixiang XUE, BEIJING (CN); Zhengyang GUO, BEIJING (CN); Tengrui ZHANG, BEIJING (CN); Yan WANG, BEIJING (CN); Yan SUN, BEIJING (CN); Yueqing GONG, BEIJING (CN)

(73) Assignee: PEKING UNIVERSITY THIRD HOSPITAL, BEIJING (CN)

(21) Appl. No.: 18/743,948

(22) Filed: Jun. 14, 2024

Related U.S. Application Data
(63) Continuation of application No. PCT/CN2021/138893, filed on Dec. 16, 2021.

(51) Int. Cl. A61K 31/496; A61K 31/501; A61P 35/00

(52) U.S. Cl. CPC A61K 31/496; A61K 31/501; A61P 35/00

(57) ABSTRACT

Provided is a pharmaceutical composition for enhancing the anti-tumor effect of an EZH2 inhibitor. The pharmaceutical composition includes an EZH2 inhibitor and an SCD1 inhibitor. The SCD1 inhibitor enhances the effect of the EZH2 inhibitor on a solid tumor. Further provided are a related preparation of the pharmaceutical composition and the use thereof in the preparation of an anti-tumor drug.

### FIELD

The invention belongs to the field of medicines, and particularly relates to an anti-tumor pharmaceutical composition comprising an EZH2 inhibitor and an SCD1 inhibitor and a use thereof in the preparation of anti-tumor drugs.

### BACKGROUND

Enhancer of zeste homolog 2 (EZH2) is the catalytic subunit of the polycomb repressive complex 2 (PRC2) and functions as a histone methyltransferase. The abnormality of EZH2 appears in many diseases (such as tumors), and several evidences show that EZH2 is related to the occurrence and progression of many cancers, as well as their poor prognosis. Overexpression of EZH2 is mainly occurred in solid tumors, including prostate cancer, breast cancer, bladder cancer, endometrial cancer, and melanoma, etc. High level of EZH2 expression is often associated with high aggressiveness, tumor progression, and poor clinical outcome and prognosis in these types of tumors.

Unlike normal cells, tumor cells directly or indirectly regulate cellular metabolic reprogramming through oncogenic mutations to meet the demands for their survival and proliferation. Epigenetic mechanisms can regulate the expression of genes involved in metabolism, thereby changing the metabolic characteristics of cells. As a key regulator of histone modification, EZH2 is involved in regulating a variety of metabolic activities of tumor cells, thus affecting the progression of cancer.

EZH2 can also promote lipid synthesis in tumor cells. In glioma cells containing telomerase reverse transcriptase (TERT) mutations, there was a positive correlation between TERT and EZH2 levels. TERT and EZH2 synergistically activate peroxisome proliferator-activated receptor gamma coactivator 1-α (PGC-1α), while the expression of fatty acid synthase (FASN) depends on PGC-1α, so that EZH2 promotes fatty acid synthesis and accumulation through TERT-EZH2 network. It has been reported that high levels of fatty acids in tumor cells can promote tumorigenesis and drug resistance by negatively regulating the DNA damage repair (DDR) pathway. In contrast, DZNep, an EZH2 inhibitor, induces lipid accumulation in nonalcoholic fatty liver cells and certain cancer cell lines, such as breast cancer. In order to clarify this difference, the role of EZH2 in adipocyte differentiation and lipid metabolism has been studied by using primary human or mouse preadipocytes and mice with specific knockout of EZH2 in adipocytes. It has been found that inhibition of EZH2 or deletion of its gene has promoted the up-regulation of Apolipoprotein E (ApoE) gene expression, which is accompanied by lipoprotein-dependent lipid absorption and ultimately leads to intracellular lipid accumulation. However, it does not affect the expression of adipocyte marker genes and adipocyte differentiation. This is contrary to the conclusion that EZH2 promotes adipogenesis and adipocyte differentiation of mouse adipose progenitor cells in previous studies. Therefore, the role of EZH2 in the regulation of lipid metabolism in tumor cells is not very clear. It is still demanded for further investigation on how EZH2 affects fatty acids, triglycerides, ketone bodies and other lipid metabolites, and what role these metabolites play in the progression of tumors.

Many efficient and selective EZH2 catalytic inhibitors have been obtained through high-throughput screening, such as EPZ005687, EI1, GSK343, GSK126 and so on, almost all of which have a 2-pyridone group in their structures. A number of EZH2 inhibitors have been developed at present as potential anti-cancer agents. Among others, CPI1205 (Lirametostat) has been tested in clinical trials, and EPZ-6438 (Tazemetostat) was approved by the FDA for the treatment of epithelioid sarcoma in 2020. However, EZH2 inhibitors are not effective in EZH2-overexpressing solid tumors, such as glioma and melanoma, which can escape the anti-tumor effect of EZH2 inhibitors through simultaneous mutations in the Ras pathway and SWI/SNF. Therefore, it has been attempted to improve the efficacy of EZH2 inhibitors by employing a therapeutic strategy combining multiple drugs or multiple anti-tumor therapies (see, e.g., Zhang Tengrui, et al., Symphony of epigenetic and metabolic regulation-interaction between the histone methyltransferase EZH2 and metabolism of tumor, Clinical Epigenetics, 2020, 12:72).

In addition, the inventor's previous research results suggested that epigenetic regulation and metabolic changes mediated by EZH2 showed a synergistic effect in cancer cells. The inventor has preliminarily found that the poor therapeutic effect of the EZH2 inhibitor may be caused by lipid metabolism disorder. Stearoyl-coenzyme A desaturase 1 (SCD1) was initially found to be associated with metabolic syndromes such as obesity, fatty liver, dyslipidemia and insulin resistance, but with the development of lipidomics and genomics, the important role of SCD1 and its product MUFA in tumors has been gradually recognized. It has been found in researches that SCD1 is closely related to the occurrence and progression of tumors, and SCD1 has become a new anti-tumor therapeutic target.

In view of the above research background, the inventor speculates that the combination of an SCD1 inhibitor may play an important role in the combination therapy of some lipid metabolism-related anti-tumor drugs, which may provide a new idea for addressing the poor therapeutic effect of EZH2 inhibitors.

### SUMMARY

Aiming at the poor activity of an EZH2 inhibitor on a solid tumor, the objective of the present invention is to provide a pharmaceutical composition comprising an EZH2 inhibitor and an SCD1 inhibitor for enhancing the anti-tumor effect of the EZH2 inhibitor, and to provide a new thought for the clinical treatment of solid tumors by means of safely, effectively, conveniently and economically using the EZH2 inhibitor.

Specifically, the present invention is implemented by the following technical solutions:

In a first aspect, the present invention provides a pharmaceutical composition for enhancing the anti-tumor effect of an EZH2 inhibitor, the pharmaceutical composition comprising an EZH2 inhibitor and an SCD1 inhibitor, wherein the SCD1 inhibitor enhances the anti-solid tumor effect of the EZH2 inhibitor.

Alternatively, in the above pharmaceutical composition, the mass ratio of the EZH2 inhibitor to the SCD1 inhibitor is 10:10 to 10:1. Substantially, the dosage ratio of the EZH2 inhibitor to the SCD1 inhibitor in the pharmaceutical composition may be determined by a clinician based on his clinical experience according to the type of cancer suffered by the patient.

Preferably, the mass ratio of the EZH2 inhibitor to the SCD1 inhibitor is selected from 10:10, 10:9, 10:8, 10:7, 10:6, 10:5, 10:4, 10:3, 10:2 or 10:1.

Alternatively, in the above pharmaceutical composition, the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, or thyroid cancer.

Preferably, the solid tumor is selected from breast cancer, melanoma, lung cancer, colon cancer, liver cancer, or gastric cancer.

Alternatively, in the above pharmaceutical composition, the EZH2 inhibitor is selected from Tazemetostat (EPZ-6438), GSK126, Lirametostat (CPI-1205), SHR2554 or PF-06821497; the SCD1 inhibitor is selected from the group consisting of CAY-10566, A939572, CVT-11127, MF-438, T-3764518, Plurisin #1, BZ36, and Abbott #7n.

Alternatively, in the above pharmaceutical composition, preferably, the EZH2 inhibitor is GSK126 and the SCD1 inhibitor is MF-438.

In a second aspect, the present invention provides a pharmaceutical formulation for enhancing the anti-tumor effect of an EZH2 inhibitor, wherein the pharmaceutical preparation is prepared from a therapeutically effective amount of the pharmaceutical composition of the first aspect above and a pharmaceutically acceptable carrier.

Alternatively, in the above pharmaceutical preparation, the pharmaceutical preparation is an oral preparation.

Preferably, the oral preparation is an oral liquid, a tablet, a powder, a capsule or a granule. In a third aspect, the present invention provides the use of the pharmaceutical composition of the first aspect or the pharmaceutical preparation of the second aspect in the preparation of an anti-tumor drug.

Alternatively, in the above use, the tumor is a solid tumor.

The solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, or thyroid cancer, and preferably, the solid tumor is selected from breast cancer, melanoma, lung cancer, colon cancer, liver cancer, or gastric cancer.

In a fourth aspect, the present invention provides the use of an SCD1 inhibitor in the preparation of a drug for enhancing the efficacy of an EZH2 inhibitor against a solid tumor.

The solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, or thyroid cancer.

Preferably, the solid tumor is selected from breast cancer, melanoma, lung cancer, colon cancer, liver cancer, or gastric cancer.

The EZH2 inhibitor is selected from Tazemetostat (EPZ-6438), GSK126, Lirametostat (CPI-1205), SHR2554 or PF-06821497.

The SCD1 inhibitor is selected from CAY-10566, A939572, CVT-11127, MF-438, T-3764518, Plurisin #1, BZ36, or Abbott #7n.

It should be understood that within the scope of the present invention, the various technical features of the present invention described above and those specifically described below (e.g., examples) may be combined with each other to form new or preferred embodiments. It will not be repeated for the sake of simplicity.

As compared with the prior art, the invention has the following beneficial effects:

The present inventors have found for the first time that the combined use of an EZH2 inhibitor and an SCD1 inhibitor can significantly enhance the efficacy of the EZH2 inhibitor in the treatment of solid tumors, and on such a basis, the invention provide a pharmaceutical composition comprising the EZH2 inhibitor and the SCD1 inhibitor, which can enhance the anti-tumor effect of the EZH2 inhibitor. Thus, the invention provides a new thought for the safe, effective, convenient and economical use of EZH2 inhibitors in the treatment of solid tumors in clinical, and has a good clinical application prospect.

### BRIEF DESCRIPTION OF THE DRAWINGS

FIG. 1: Hematological tumors are more sensitive to GSK126 than solid tumors. (A) Proliferation of B16F10, Huh7 and SMMC7721 cells in GSK126 (10-20 μM) treatment group was monitored by Incucyte S3, with 6 replicates per group; (B) viability of Daudi and THP-1 cells after GSK126 (200 nM-24 μM) treatment for 24 h and 48 h was measured by CCK-8 assay, with 5 replicates per group; (C) western blot analysis (WB) of B16F10 cells 48 h after the treatment without or with GSK126 (6 μM, 12 μM), with H3 used as an internal reference for histone. 5 to 6 replicates were established for each group, with *P<0.05, **P<0.01, and ***P<0.001, and ns for no statistical difference.

FIG. 2: Melanoma cells B16F10 are less sensitive to GSK126. (A) Cell scratch assay, showing the effect of GSK126 (10 μM) treatment on the migration capability of B16F10 cells, with 3 replicates per group; (B-C) the effect of GSK126 (10, 13, and 15 μM) on the apoptosis rate of B16F10 cells, as examined by flow cytometry (C) and Annexin V staining assay. 3 replicates were established for each group, with *P<0.05, **P<0.01, and ***P<0.001, and ns for no statistical difference.

FIG. 3: GSK126 results in significant elevated levels of 6 fatty acids in melanoma B16F10 cells.

FIG. 4: Lipid accumulation in mice after GSK126 treatment. (A) H & E and oil red O staining of the liver tissue sections of the tumor-bearing mice in the control group and the GSK126 treatment group, in which the statistical results of negative or positive staining area were shown on the right (3 replicates per group); (B) TG level in the serum of the tumor-bearing mice in the control group and the GSK126 treatment group (5 replicates per group); (C-D) mRNA (C) and protein (D) levels of the genes related to fatty acid anabolism in the GSK126-treated HCC cells, as detected by RT-qPCR and WB (3 replicates per group). 3 to 5 replicates were established for each group, with *P<0.05, **P<0.01.

FIG. 5: Effect of GSK126 on the metabolic profile of Daudi cells. (A) Orthogonal partial least squares discriminant analysis (OPLS-DA) score plot based on metabolic analysis (6 replicates per group). C: control, G: GSK126 treatment. (B) Score plot for validating the OPLS-DA model (R2Y=0.973; Q2Y=0.931). (C) The number of metabolites of each category in metabolomics. (D) Human Protein Atlas (HPA) database analysis reveals SCD protein expression levels in different cancer patients. (E) Viability of Daudi cells after palmitic acid treatment was analyzed by CCK-8 kit (3 replicates per group). 3-6 replicates per group, *P<0.05, **P<0.01.

FIG. 6: The combination with an SCD1 inhibitor enhances the inhibitory effect of GSK126 on cancer cells. (A) Differential metabolites between DMSO and GSK126-treated Daudi cells. The heat map shows the proportional abundance of 71 differential metabolites with VIP (variable weight value) greater than 1. Metabolite categories are shown in different colors. (B) Four significantly reduced fatty acids in GSK126-treated Daudi cells. (C) The mRNA levels of lipid metabolism genes in GSK126-treated Daudi cells were detected by RT-qPCR (3 replicates per group). (D) Viability of GSK126 and palmitinic acid-treated Daudi cells was analyzed by CCK-8 assay kit (5 replicates per group). (E) Viability of GSK126 and stearic acid-treated Daudi cells was analyzed by CCK-8 assay kit (5 replicates per group). (F) Proliferation curves of B16F10 and SMMC7721 cells treated with GSK126 and MF-438 were monitored by IncuCyte S3 (5 replicates per group). (G) CCK-8 assay kit detects the viability of B16F10 and SMMC7721 cells treated with GSK126 and MF-438. CI is calculated by CompuSyn software. CI<0.9 indicates synergism and CI>1.1 indicates antagonism. (H) Schematic diagram of mouse tumor-bearing experiment. (I-J) Tumor weight (I) and tumor growth curve (J) of the mice loaded with B16F10 cells. The tumor volume was measured with a vernier caliper every 2 days. Tumor volume=(length*width*width)/2. (K) Representative tumor pictures of all experimental groups. 3-8 replicates per group, *P<0.05, **P<0.01, ***P<0.001.

### DETAILED DESCRIPTION

The inventor discovers for the first time that the efficacy of the EZH2 inhibitor for the treatment of the solid tumor can be significantly enhanced by the combination of the EZH2 inhibitor and the SCD1 inhibitor through a large amount of screening experiments after the thorough investigation on the regulatory mechanism of the EZH2 on the lipid metabolism in the tumor cells and the anti-tumor mechanism of the EZH2 inhibitor. The present invention has been accomplished on such a basis.

As used herein, the EZH2 inhibitor and the SCD1 inhibitor in the pharmaceutical composition of the present invention may be administered in the same pharmaceutical preparation or in different pharmaceutical preparations. In the case of administration in different pharmaceutical preparations, the dosage forms of the EZH2 inhibitor and the SCD1 inhibitor can be the same or different. Moreover, the EZH2 inhibitor and the SCD1 inhibitor may be administered simultaneously or sequentially.

As used herein, “SCD1” is a key hub that regulates the lipid composition in tumor cells and also plays an important role in signaling pathways for tumor cell growth, survival, and malignant transformation. Inhibitors targeting SCD1 can inhibit the proliferation of tumor cells, induce apoptosis and reverse the chemoresistance of tumor cells, and show certain anti-tumor activity in preclinical experiments. In the present invention, the “SCD1 inhibitor” is selected from CAY-10566, A939572, CVT-11127, MF-438, T-3764518, Plurisin #1, Z36 or Abbott #7n.

Non-limiting examples of tumors treatable with the pharmaceutical compositions of the present invention may include, but are not limited to, biliary tract cancer (e.g., cholangiocarcinoma), bladder cancer, breast cancer (e.g., breast adenocarcinoma, papillary breast cancer, breast cancer, medullary breast cancer, triple negative breast cancer, HER2 negative breast cancer, HER2 positive breast cancer, male breast cancer, advanced metastatic breast cancer, progesterone receptor negative breast cancer, progesterone receptor positive breast cancer, relapsed breast cancer), brain cancer (e.g., meningioma; gliomas such as astrocytomas, oligodendrogliomas; medulloblastoma), bronchial cancer, cervical cancer (e.g., cervical adenocarcinoma), choriocarcinoma, colorectal cancer (e.g., colon cancer, rectal cancer, colorectal adenocarcinoma), epithelial cancer, endometrial cancer (e.g., uterine cancer, uterine sarcoma), esophageal cancer (e.g., esophageal adenocarcinoma, Barrett's adenocarcinoma), Ewing's sarcoma, ocular cancer (e.g., intraocular melanoma, retinoblastoma), gallbladder cancer, gastric cancer (e.g., gastric adenocarcinoma), gastrointestinal stromal tumor (GIST), glioblastoma multiforme, head and neck cancer (e.g., head and neck squamous cell carcinoma, oral cancer (e.g., oral squamous cell carcinoma (OSCC)), throat cancer (e.g., laryngeal cancer, pharyngeal cancer, rhinopharynx cancer, oropharyngeal cancer)), kidney cancer (e.g., nephroblastoma, also known as Wilms' tumor, renal cell carcinoma), liver cancer (e.g., hepatocellular carcinoma (HCC), malignant liver cancer), lung cancer (e.g., bronchial carcinoma, small cell lung carcinoma (SCLC), non-small cell lung carcinoma (NSCLC), lung adenocarcinoma), leiomyosarcoma (LMS), myelodysplastic syndrome (MDS), mesothelioma, neuroendocrine carcinoma (e.g., gastroenteropancreatic neuroendocrine tumor (GEP-NET), carcinoid tumor), osteosarcoma, ovarian carcinoma (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), pancreatic islet cell tumor), penile cancer (e.g., Paget's disease of the penis and scrotum), prostate cancer (e.g., prostatic adenocarcinoma), rectal cancer, rhabdomyosarcoma, skin cancer (e.g., squamous cell carcinoma (SCC), corneal acanthoma (KA), melanoma, basal cell carcinoma (BCC)), small intestine carcinoma (e.g., appendiceal carcinoma), soft tissue sarcoma (e.g., malignant fibrous histiocytoma (MFH), liposarcoma, chondrosarcoma, fibrosarcoma), sebaceous gland carcinoma, sweat gland carcinoma, synovioma, testicular carcinoma (e.g., seminoma, testicular embryonal carcinoma), thyroid carcinoma (e.g., thyroid papillary carcinoma, papillary thyroid carcinoma (PTC), medullary thyroid carcinoma), urethral carcinoma, vaginal carcinoma, and vulvar carcinoma (e.g., vulvar Paget's disease).

As used herein, the dosage forms of the pharmaceutical preparation of the present invention is selected from the group consisting of tablets, capsules, granules, oral liquid or inhalant. Preferably, the dosage form of the present invention is a tablet or a capsule.

As used herein, the “pharmaceutically acceptable carrier” of the present invention refers to a conventional pharmaceutical carrier in the field of pharmaceutical preparation, which is selected from one or more of a filler, a binder, a disintegrant, a lubricant, a suspending agent, a wetting agent, a pigment, a flavoring agent, a solvent, and a surfactant.

The filler of the invention includes, but not limited to, starch, microcrystalline cellulose, sucrose, dextrin, lactose, powdered sugar, glucose and the like; the lubricant includes, but not limited to, magnesium stearate, stearic acid, sodium chloride, sodium oleate, sodium lauryl sulfate, poloxamer and the like; the binder includes, but is not limited to, water, ethanol, starch slurry, syrup, hydroxypropyl methyl cellulose, sodium carboxymethyl cellulose, sodium alginate, polyvinylpyrrolidone, and the like; the disintegrant includes, but is not limited to, starch effervescent mixture, namely sodium bicarbonate and citric acid, tartaric acid, low-substituted hydroxypropyl cellulose, and the like; the suspending agent includes, but is not limited to, polysaccharides such as gum acacia, agar, alginic acid, cellulose ether, carboxymethyl chitosan, and the like; and the solvent includes, but are not limited to, water, equilibrated salt solutions, and the like.

Preferably, the drug of the present invention can be prepared into various solid oral preparations, liquid oral preparations, and the like. The pharmaceutically acceptable oral solid preparations include ordinary tablets, dispersible tablets, enteric coated tablets, granules, capsules, dropping pills, pulvis, and the like. The oral liquid preparations include oral liquid, emulsion, and the like. Alternatively, the drug of the present invention may be prepared into a topical dosage form, such as an inhalant, and the like.

The various dosage forms described above can be prepared according to the conventional techniques in the field of pharmaceutical preparation.

In the pharmaceutical composition, the pharmaceutical preparation and the medical use described above, the administration time, number, and frequency and the like of the “EZH2 inhibitor” and the “SCD1 inhibitor” need to be determined according to the specific diagnosis of the diseases, which is well within the scope of the skills of those skilled in the art.

The invention is further described below with reference to specific examples. It should be understood that the specific examples described herein are merely illustrative of the invention, and are not intended to limit the scope of the invention.

If no specific technology or conditions are specified in the examples, the technology or conditions described in the literatures in this field or in the product specification shall be followed. If no manufacturer is indicated for the reagent or instrument used, it is a conventional product that can be purchased through regular channels.

The experimental methods in the following examples are all conventional methods, unless otherwise specified. The test materials used in the following examples are commercially available, unless otherwise specified.

The percentages and parts referred to in the present invention are weight percentages and weight parts, unless otherwise stated.

### EXAMPLES

1. Many Solid Tumor Cell Lines have Low Sensitivity to EZH2 Inhibitor GSK126.

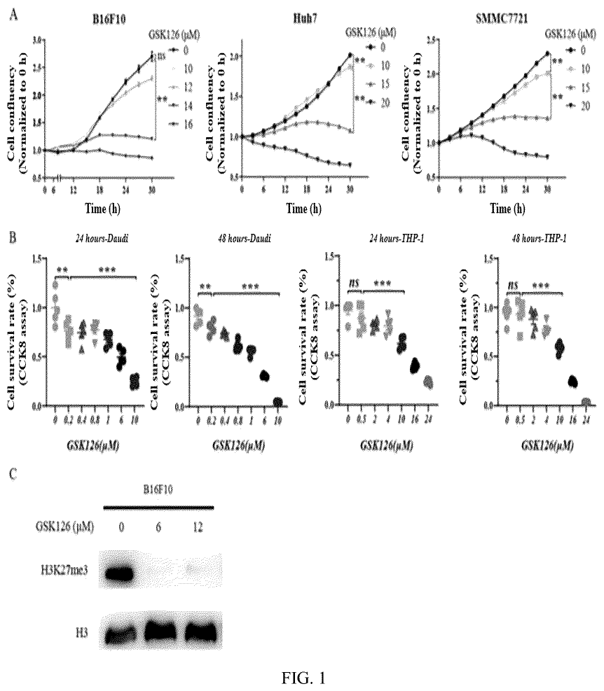

In order to accurately assess the responsiveness of solid tumor cells to EZH2 inhibitors, the effects of GSK126 at different concentrations on the proliferation of melanoma B16F10 cells and hepatoma Huh7 and SMMC-7721 cells were initially detected by IncuCyte S3×Live-Cell Analysis System. The results showed that GSK126 inhibited cell proliferation in a dose-dependent manner, whereas GSK126 at a higher concentration of 10 μM was less effective (section A of FIG. 1).

Under the same conditions, the results of CCK-8 cell proliferation assay showed that the proliferation of hematological tumor cells Daudi and THP-1 was significantly inhibited after treated with 10 μM GSK126 for 24 h and 48 h. In particular, Burkitt lymphoma cells (Daudi) were already very sensitive to 1 μM GSK126 (section B of FIG. 1). As consistent with other studies, we have found that solid tumor cell lines are generally insensitive to EZH2 inhibitors, whereas many studies have shown that the IC₅₀ (half-maximal inhibitory concentration) of most hematologic cancer cell lines treated with GSK126 is lower than 1 μM.

Moreover, it was observed that the H3K27me3 level in B16F10 cells was significantly down-regulated by GSK126 at 6 μM (section C of FIG. 1), but the proliferation of cancer cells could not be effectively inhibited by GSK126 at 10 μM (section A of FIG. 1). This indicates that GSK126 can not exert its anti-tumor effect, when it has been able to effectively inhibit the histone methyltransferase activity of EZH2.

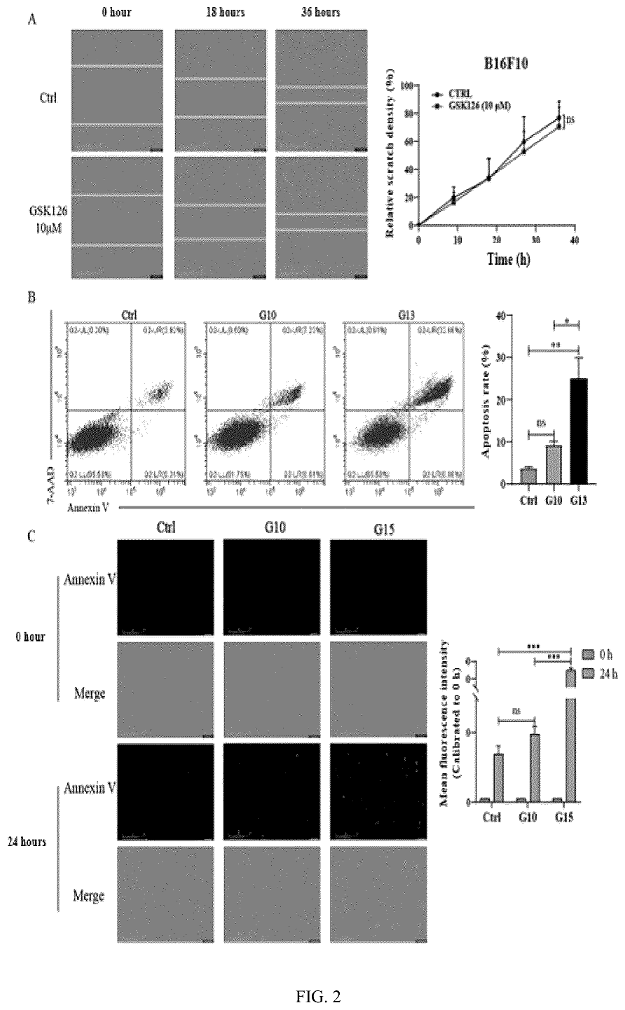

Next, to further validate the effect of GSK126 at 10 μM on melanoma cells B16F10, scratch assay was conducted. Similar to the cell proliferation assay, after 36 h of culture, GSK126 did not effectively inhibit cell migration at 10 μM as compared with the control group (section A of FIG. 2). In addition, to assess the anti-survival effect of GSK126, B16F10 cells were treated by GSK126 at different concentrations for different durations, and the apoptosis rate of the cells was analyzed by flow cytometry (section B of FIG. 2) and live-cell fluorescence imaging (section C of FIG. 2). The results indicate that GSK126 at 10 μM has a limited capability to induce apoptosis in B16F10 cells. Accordingly, the proliferation of solid tumor cells (B16F10, Huh7 and SMMC7721) and the migration and survival of B16F10 cells were not significantly inhibited after treated by GSK126 at higher concentration (10 μM).

### 2. GSK126 Up-Regulates the Fatty Acid Levels in Melanoma B16F10 Cells

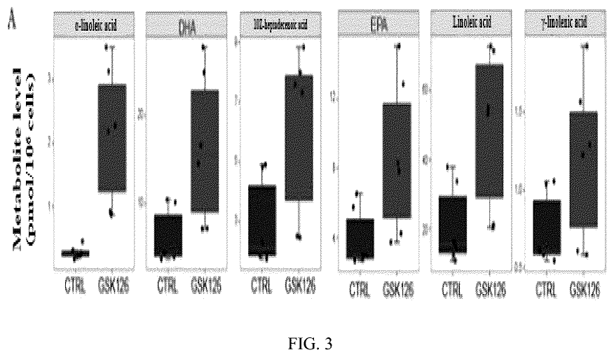

Among the metabolites with significant changes, the levels of many fatty acids increased in the GSK126 group. There are five polyunsaturated fatty acids, including α-linolenic acid, DHA, EPA, linoleic acid, and γ-linolenic acid) and one monounsaturated fatty acid, i.e., 10Z-heptadienoic acid (FIG. 3).

This indicates that GSK126 can improve the fatty acid abundance. These fatty acids can be used as substrates for lipid synthesis, form structures of plasma membrane, but also inhibit cell growth and induce apoptosis when excessively accumulated. De novo synthesis of fatty acids can provide raw materials for the biofilm structure, energy production and protein modification of cancer cells. Moreover, high LD and cholesteryl ester content in tumors are correlated with cancer invasiveness. This suggests that lipid accumulation may be responsible for the poor anti-tumor efficacy of GSK126.

### 3. Regulation of Lipid Metabolism can Enhance the Inhibitory Effect of GSK126 on Cancer Cells

### 3.1 GSK126 Treatment Leads to Lipid Accumulation in Mice

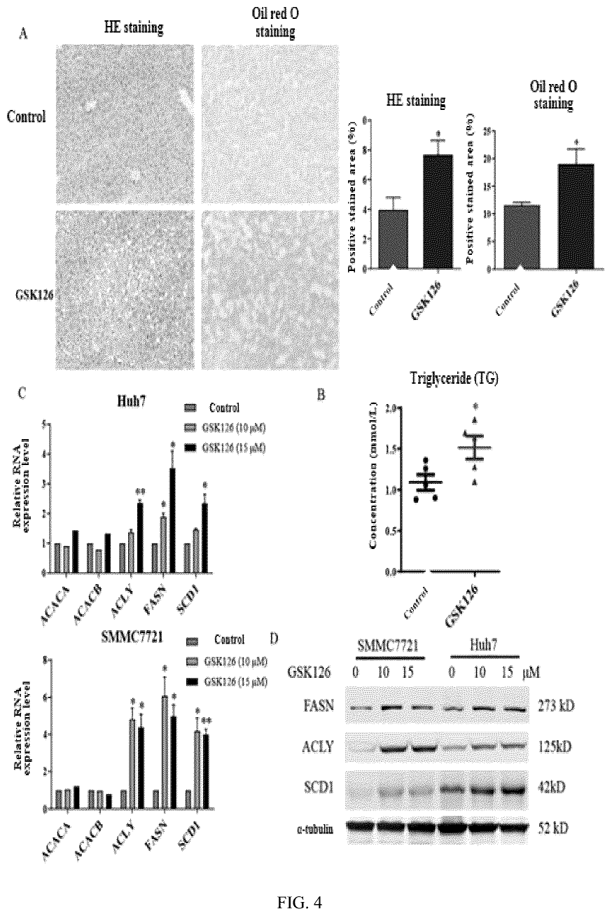

Since a plurality of fatty acids were up-regulated in the B16F10 cells treated with GSK126, alterations in lipid metabolism in the tumor-bearing mice were explored. C57BL/6 mice of 6-8 weeks old were subcutaneously implanted with B16F10 cells and treated with GSK126 again. H & E and Oil Red O staining of the liver tissue sections from the mice showed a significant increase in fat vacuoles and lipid deposition in the GSK126 treatment group (section A of FIG. 4). The levels of triglycerides (TG) in the blood of the mice were also significantly elevated (section B of FIG. 4). These results suggest that GSK126 can regulate lipid metabolism in the liver and throughout the body. Therefore, the expression of the fatty acid synthesis-related genes in GSK126-treated hepatocellular carcinoma (HCC) cell lines SMMC7721 and Huh7 was further detected. As shown in sections C and D of FIG. 4, the mRNA and protein levels of ACLY, FASN, and SCD were significantly elevated under GSK126 treatment, which is consistent with the RNA-seq data.

### 3.2 Regulation of Lipid Metabolism can Enhance the Inhibitory Effect of GSK126 on Cancer Cells

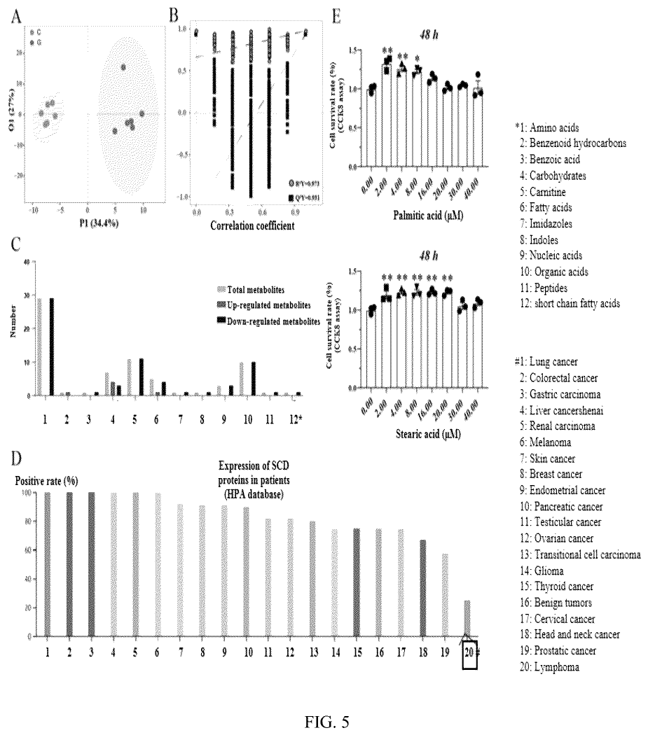

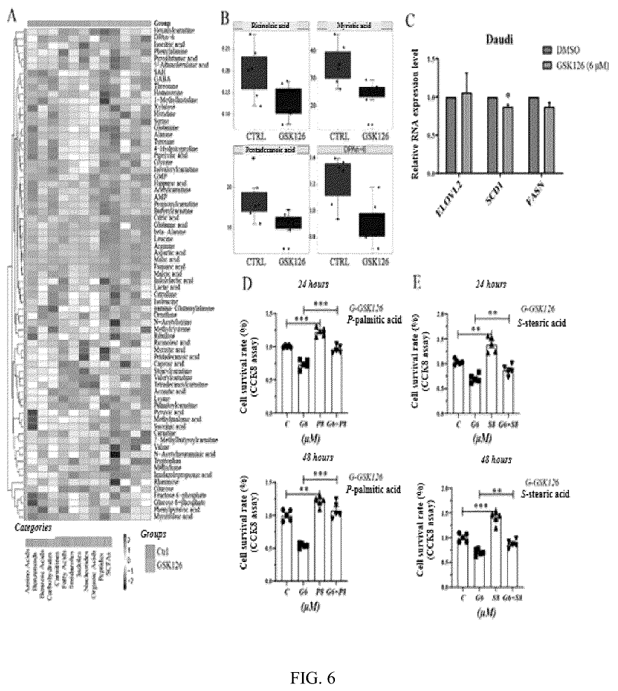

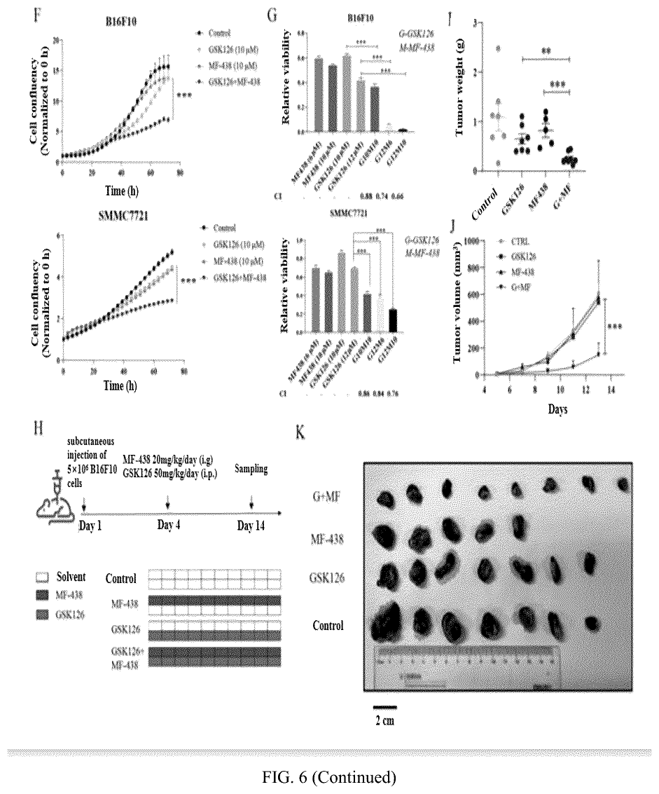

In order to verify that the lipid metabolism of cancer cells is the key factor affecting the sensitivity of the EZH2 inhibitor, UPLC-MS/MS system was used to analyze the metabolomics of the Daudi cells treated with GSK126 at a concentration of 6 μM (close to the IC₅₀ value). The differences in the metabolome between the cells in the control group and the GSK126 treatment group were obvious by OPLS-DA analysis (sections A and B of FIG. 5). There were 71 metabolites with significant changes and VIP values greater than 1.0. There were 65 up-regulated and 6 down-regulated metabolites in the GSK126-treated cells as compared to the control (section A of FIG. 6 and section C of FIG. 5). Among these significantly altered metabolites, we found four fatty acids decreased in the GSK126-treated cells, including one MUFA (ricinoleic acid) and one PUFA (DPA n-6) (section B of FIG. 6). This indicates that the basal lipid metabolism in hematological tumor cells is weaken after the GSK126 treatment. According to the analysis of The Human Protein Atlas database, the positive rate of SCD1 protein expression in lymphoma is lower than that in other solid tumors (section D of FIG. 5), which indirectly indicates that the basal lipid metabolism of hematological tumor cells is not high.

Consistent with the metabolomics results, GSK126 treatment did not upregulate the expression of ELOVL2, SCD1, and FASN (section C of FIG. 6). In addition, it was examined whether fatty acid supplementation decreased the sensitivity of Daudi cells to GSK126 or not. Cell proliferation was moderately promoted by the addition of palmitic acid and stearic acid (section E of FIG. 5), and the fatty acid supplementation recovered the cell viability inhibited by GSK126 to varying degrees (sections D and E of FIG. 6). These results suggest that the increased fatty acid levels in the cancer cells may impair the anti-tumor effect of the EZH2 inhibitor.

Our findings indicate that ELOVL2 and SCD1 are directly regulated by EZH2 and H3K27me3 in the promoter region, suggesting that ELOVL2 or SCD1 inhibition may enhance the cellular sensitivity to the EZH2 inhibitor. Because SCD1 catalyzes the desaturation of the saturated fatty acids, upstream of PUFA extension, the combination therapy with the SCD1 inhibitor MF-438 was used in subsequent studies.

The brief steps of the relevant in vitro and in vivo experiments are as follows:

### In Vitro Experiment:

(1) Cells in the logarithmic growth phase were digested with 0.25% trypsin and the digestion was terminated by the addition of serum-containing medium. It was then centrifuged at 200 g for 5 min;
(2) The single cell suspension at the corresponding concentration was prepared by a microscopic counting method, and then added into a 96-well culture plate, so that the volume of the culture medium in each well was 150 μL (about 5000 cells). 5-6 duplicate wells were set for each treatment group;
(3) The 96-well culture plate was placed in a cell incubator and further cultured for about 12-24 h. When the confluence in each well was observed to about 20-30% under the microscope, the culture medium was discarded. Then, according to the experiment requirements, the culture medium containing drugs was added, and groups with a series of drug concentrations were set (for example, when B16F10 cells and SMMC7721 cells were treated, the concentration of GSK126 was set at 10 μM, and the concentration of MF-438 was set at 10 μM). 5-6 duplicate wells were set for each treatment group;
(4) The 96-well culture plate was placed in the detector in the incubator and tested with IncuCyte S3® Live-Cell Analysis System. Four sets of phase contrast images of different areas in each hole were taken at a 3 h interval using a 10×objective lens.
(5) After real-time monitoring for 72 h, IncuCyte S3 image analysis software was used to detect cell edge so as to analyze and determine the percentage of confluence of the cells in each well, which was then used to evaluate cell proliferation.

### In Vivo Experiment:

(1) Single cell suspension of the tumor cells (B16F10) in logarithmic growth phase was prepared. The cell concentration was adjusted to 5.0×10⁶ cells/mL using sterile PBS as solvent.
(2) Mice (female C57BL/6 mice, 6-8 weeks of age, weighing 18-20 g) were anesthetized with sodium phenobarbital (40 mg/kg) and shaved to facilitate tumor bearing and tumor measurement in a superclean bench;
(3) B16F10 cells (0.1 mL of single cell suspension) were subcutaneously implanted into the upper right area of the back of the mouse with a syringe, and an obvious spherical bulge was formed at the injection site;
(4) The mice were returned to the rearing cage, and then observed for 1-2 days;
(5) When the tumor (about 50 mm³) appeared on the back of the mice, they were randomly divided into groups (the control group and the treatment group, 5-6 mice/group) and treated with drugs;
(6) The treatment groups were divided into the GSK126 alone group (50 mg/kg/day), the MF-438 alone group (20 mg/kg/day) and the combination group of GSK126 (50 mg/kg/day) and MF-438 (20 mg/kg/day). Drugs used in the treatment, including GSK126 and MF-438, were purchased from Shanghai Lanmu Chemical Co., Ltd., and the solvent was formulated according to the instruction, while the control group was treated with the same amount of solvent. GSK126 was administered by intraperitoneal injection, and MF-438 was administered by intragastric administration. The length (a) and width (b) of the tumor as well as the changes of the body weight of the mice were recorded with a vernier caliper and a weighing balance every 2 days.
(7) After 14 days of treatment, the mice were sacrificed and the corresponding tissue samples were preserved for downstream experiments.

The results showed that the combination of GSK126 and MF-438 significantly inhibited the proliferation of B16F10 and SMMC7721 cells as compared to the treatment using GSK126 or MF438 alone for 72 hours (section F of FIG. 6). CI analyzed by CCK-8 assay and CompuSyn software showed the synergistic effect of GSK126 and MF-438 at different concentrations in both cells (section G of FIG. 6). In vivo experiments showed that the pretreatment of B16F10 cells with the combination of drugs significantly reduced the growth of subcutaneously implanted tumors in mice (sections H-K of FIG. 6).

In conclusion, the above results suggest that the inhibition of SCD1 can enhance the anti-cancer effect of the EZH2 inhibitor. The invention provides a new thought for using the EZH2 inhibitor to treat solid tumors safely, effectively, conveniently and economically in clinical, and has a good clinical application prospect.

It will be apparent to those skilled in the art that various modifications and variations can be made in the present invention without departing from the spirit and scope of the invention. Thus, the present invention is intended to include such modifications and variations provided they come within the scope of the appended claims and their equivalents.

1. A pharmaceutical composition for enhancing the anti-tumor effect of an EZH2 inhibitor, comprising:
an EZH2 inhibitor and an SCD1 inhibitor,
wherein the SCD1 inhibitor enhances the anti-solid tumor effect of the EZH2 inhibitor.

2. The pharmaceutical composition according to claim 1, wherein the mass ratio of the EZH2 inhibitor to the SCD1 inhibitor is 10:10-10:1.

3. The pharmaceutical composition according to claim 1, wherein:
the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, or thyroid cancer, and preferably, the solid tumor is selected from breast cancer, melanoma, lung cancer, colon cancer, liver cancer, or gastric cancer.

4. The pharmaceutical composition according to claim 1, wherein:
the EZH2 inhibitor is selected from Tazemetostat (EPZ-6438), GSK126, Lirametostat (CPI-1205), SHR2554 or PF-06821497; the SCD1 inhibitor is selected from CAY-10566, A939572, CVT-11127, MF-438, T-3764518, Plurisin #1, BZ36, and Abbott #7n.

5. The pharmaceutical composition according to claim 1, wherein the EZH2 inhibitor is GSK126, and the EZH2 inhibitor is MF-438.

6. A pharmaceutical preparation for enhancing the anti-tumor effect of an EZH2 inhibitor, wherein the pharmaceutical preparation is prepared from a therapeutically effective amount of the pharmaceutical composition according to claim 1 and a pharmaceutically acceptable carrier.

7. The pharmaceutical preparation according to claim 6, wherein the pharmaceutical preparation is an oral preparation, and preferably, the oral preparation is an oral liquid, a tablet, a powder, a capsule or a granule.

8. Use of the pharmaceutical composition according to claim 1 in the preparation of an anti-tumor drug.

9. The use according to claim 8, wherein:
the tumor is a solid tumor; and
the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, or thyroid cancer, and preferably, the solid tumor is selected from breast cancer, melanoma, lung cancer, colon cancer, liver cancer, or gastric cancer.

10. Use of an EZH2 inhibitor for the preparation of a drug for enhancing the efficacy of the EZH2 inhibitor against a solid tumor, wherein the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, or thyroid cancer, and preferably, the solid tumor is selected from breast cancer, melanoma, lung cancer, colon cancer, liver cancer, or gastric cancer; the EZH2 inhibitor is selected from Tazemetostat (EPZ-6438), GSK126, Lirametostat (CPI-1205), SHR2554 or PF-06821497; the SCD1 inhibitor is selected from CAY-10566, A939572, CVT-11127, MF-438, T-3764518, Plurisin #1, BZ36, and Abbott #7n.
